(19) United States
(12) Patent Application Publication
(10) Pub. No.: US 2006/0251673 A1
(43) Pub. Date: Nov. 9, 2006

(54) CULTIVATION METHOD AND APPLICATIONS FOR ANTRODIA CAMPHORATA

(76) Inventors: San-Bao Hwang, Taipei County (TW); Yu-Lung Wang, Yunlin County (TW); Sheng-Yung Liu, Taipei County (TW)

Correspondence Address: JIANQ CHYUN INTELLECTUAL PROPERTY OFFICE, 7 FLOOR-1, NO. 100, ROOSEVELT ROAD, SECTION 2, TAIPEI, 100, TW

(21) Appl. No.: 11/308,795

(22) Filed: May 5, 2006

(30) Foreign Application Priority Data
May 6, 2005 (TW) 94114665

(51) Int. Cl. A61K 36/06; A01G 1/04

(52) U.S. Cl. 424/195.15; 47/1.1

(57) ABSTRACT

A method for cultivating Antrodia Camphorata is provided. First, Antrodia Camphorata is pre-incubated on a sloped surface culture medium. A portion of the pre-incubated Antrodia Camphorata is transferred to a liquid culture medium for incubation. Then, Antrodia Camphorata incubated in the liquid culture medium is moved to a solid culture medium for incubation. The cultivated Antrodia Camphorata has similar efficacies for inhibiting cancel cell growth, reducing the occurrence of atherosclerosis or recovering the damaged liver functions, as the wild Antrodia Camphorata.

### CROSS-REFERENCE TO RELATED APPLICATION

This application claims the priority benefit of Taiwan application serial no. 9411 4665, filed on May 6, 2005. All disclosure of the Taiwan application is incorporated herein by reference.

### BACKGROUND OF THE INVENTION

1. Field of the Invention

The present invention generally relates to a cultivation method and the application of fungi. More particularly, the present invention relates to a cultivation method and the applications of Antrodia camphorata.

2. Description of Related Art

Antrodia camphorata, also known as “niu-chang-chiu” or “niu-chang-ku”, is classified as Aphyllophorales, Polyporaceae, which is a perennial fungi and a special fungus which grows only in Taiwan. It is raised on Cinnamomum kanehirai, which is a unique plant found in Taiwan. Antrodiac camphorata is a precious and probably the most expensive medicinal fungus in existence. It was found as a new species in 1990; and it was named Antrodia cinnamomea in 1995 for the second new species announcement; it was renamed as Antrodia camphorata during the third announcement for the new species in 1997.

There are three major triterpenoids (antcin A, antcin B, and antcin C) extracted from the fruit body of Antrodia camphorata, discovered in previous studies. Later on, three more new triterpenoids: antrocin, 4,7-dimethoxy-5-methyl-1,3-benzodioxole, and 2,2′,5,5′-tetramethoxy-3,4,3′,4′-bimethyl-enedioxy-6,6′-dimethylbiphenyl are found in the extract of the fruit body from the Antrodia camphorata. Another four new triterpenoids including antcin E, antcin F, methylantcinate G, and methylantcinate H are found in 1996. Also in 1996, two new compounds zhankuic acid D and zhankuic acid E, that are compounds having the backbone structure of ergostane and extracted from the fruit body of the Antrodia camphorata, are discovered along with three new compounds having the backbone structure of lanostane including 15α-acetyl-dehydro-sulphurenic acid, dehydroeburicoic acid, and dehydro-sulphurenic acid.

The components in the compositions of Antrodia camphorata are numerous and complicated. In addition to the triterpenoids, there are many other biological active materials, such as polysaccharides, SOD, adenosine, low molecular weight proteins, vitamins, minor elements, creatines, steroides, blood pressure stabilizer . . . etc. The unique triterpenoid compounds obtained from the Antrodia camphorata are proven to be effective in the inhibition of cancer cells growth and the activation of neuron cells. However, more studies are required to further investigate the mechanisms for the abovementioned activities.

As cultivated in the dark, moist environment at medium lower altitudes, the wild Antrodia camphorata grows slowly (about 6-10 months). Consequently, the fruit body will take a longer time to reach maturity. The “niu-chang-chiu” and Antrodia camphorata are nationally protected plants, and mass production of Antrodia camphorata by artificial cultivation is not yet successful. Due to the unlawful collection, Antrodia camphorata is near to extinction.

In U.S. Pat. No. 6,558,943 B1, Li et al. disclosed the solid state cultivation of Cordyceps sinesis. In US Publication No. 2003/0138408 A1, Lan et al. disclosed the cultivation of the Antrodia camphorata in isolated plastic bags. In U.S. Pat. Nos. 6,391,615 B1, 6,395,271 B1and 6,355,475 B1, Huang et al. disclosed the isolation of Antrodia camphorata from cultivation medium. In US Publication No. 2003/0086908 A1, Wu et al. disclosed the using of stimulations and different PHs for tuning the best conditions for the Antrodia camphorata. In US Publication No. 2003/0148517 A1, Chen et al. disclosed biological tests for Antrodia camphorata. In US Publication No. 2003/0113297 A1, Chen et al. disclosed the physiological tests of Antrodia camphorata for liver cancer.

In J. Agric. Food Chem, Song et al. disclosed the model for testing the efficacy of applying Antrodia camphorata to treat liver cancer(T-Y Song and G-C Yen, “Protective Effects of Fermented Filtrate from Antrodia camphorata in submerged Culture against CCl₄-induced Hepatic Toxicity in Rats,” J. Agric. Food Chem., 2003, 51,1571-1577); the model for testing anti-oxidant properties of Antrodia camphorata is also developed (T-Y Song and G-C Yen, “Antioxidant Properties of Antrodia camphorata in Submerged Culture,” J. Agric. Food Chem., 2002, 50,3322-3327. In the final report of the research for health food by Prof Yang Hsin-Ling of the Nutritional Dept. of Chinese Medicinal College, it is published in “The cardiovascular protection of Antrodia camphorata on endothelium tissue is investigated”.

### SUMMARY OF THE INVENTION

Broadly speaking, the present invention provides a cultivation method for Antrodia camphorata in replace of the collection of the wild Antrodia camphorata. Besides, the invention provides a mass production method for producing the mycelium of the Antrodia camphorata with similar biological effects as the wild Antrodia camphorata.

In accordance with one aspect of the present invention, the present invention is directed to a food composition comprising the Antrodia camphorata cultivated by the method of the present invention.

In accordance with another aspect of the present invention, the present invention is directed to a pharmaceutical composition comprising the Antrodia camphorata cultivated by the method of the present invention.

In accordance with yet another aspect of the present invention, the present invention is directed to cosmetic products comprising the Antrodia camphorata cultivated by the method of the present invention.

In accordance with again another aspect of the present invention, the present invention is directed to a soft drink comprising the Antrodia camphorata cultivated by the method of the present invention.

In accordance with yet again another aspect of the present invention, present invention is directed to a fermentation method to produce alcoholic products from the cultivated Antrodia camphorata, where the anti-oxidation efficacy of the alcoholic extracts from the cultivated Antrodia camphorata is 2.5-4.0 times relative to the non-fermented product (without alcohol) from the cultivated Antrodia camphorata.

According to the present invention, a solid state cultivation method is proposed. A pre-cultivation process for the Antrodia camphorata on a slope surface is performed, following by transferring a portion of the pre-cultivated Antrodia camphorata into a liquid culture medium. Thereafter, the Antrodia camphorata cultivated in the liquid culture medium is transferred to a solid culture medium.

According to the preferred embodiment of the present invention, the above mentioned solid culture medium including a substrate, a carbon source, a nitrogen source and at least an inorganic salt. The above mentioned substrate is selected from the group consisting of rice, agar, or a mixture of rice and agar. In one of the embodiment, the rice is selected from the group consisting of Glutinous Rice, Japonica Rice, Indica Rice, or Zizania also called Water Rice or Wild Rice).

According to the preferred embodiment of the present invention, if the substrate is made from the mixture of rice and agar, with the weight percentage of the rice ranging from 0% to 60%, and the weight percentage of agar ranging from 0% to 3%, as the total weight of the solid culture medium being 100%.

According to the preferred embodiment of the present invention, the above mentioned carbon source is selected from the group consisting of glucose (also as Dextrose), fructose, galactose, lactose, starch, cellulose, and sucrose.

According to the preferred embodiment of the present invention, the above mentioned nitrogen source is selected from peptone, tripeptone, beef extracts, malt extracts, or yeast extracts.

According to the preferred embodiment of the present invention, the above mentioned inorganic salts are selected from the group consisting of phosphates, sodium chloride (NaCl), magnesium sulfate (MgSO₄),and potassium chloride (KCl); the concentration of the inorganic salts are ranged from 0.01% to 2.5%; and the phosphates is selected from the group consisting of di-potassium hydrogen phosphate (K₂HPO₃), potassium phosphate (K₃PO₃), potassium di-hydrogen phosphate (KH₂PO₃), sodium di-hydrogen phosphate (NaH₂PO₃), di-sodium hydrogen phosphate (Na₂HPO₃), and the concentration of the phosphates is ranged from 0.01% to 1.0%.

According to the preferred embodiment of the present invention, the above mentioned, the Antrodia camphorata cultivated from the solid culture media is applied to inhibit the growth of human cancer cells. Besides, the Antrodia camphorata has the auxiliary activity to inhibit the growth of human cancer cells, such as breast cancer cells (such as MCF-7), liver cancer cells (such as Hep 3B), prostate cancer cells (such as LNCaP), and the other cancer cells. Moreover, possible application of the cultivated Antrodia camphorata includes the inhibition for the oxidation of the human low density lipoproteins, the reduction for the atherosclerosis and the recovery of the liver damages.

As above mentioned food composition contains the Antrodia camphorata cultivated by the method of the present invention, the form of the food composition, according to the preferred embodiment, can be as powders, gels, solid or liquid states. In addition, the above mentioned food composition further includes a food additive, which is selected from the group consisting of the Chinese herbal medicine (or herbals), a healthy food, and food materials.

As above mentioned pharmaceutical composition contains the Antrodia camphorata cultivated by the method of the present invention, the form of the pharmaceutical composition, according to the preferred embodiment, is selected from the group consisting of tablet, powder, granular, capsule, rapid (oral integrated) tablet, injection, lyophilized injection, suspension, emulsion, syrup, tincture or solution. An acceptable second therapeutic agent can be added at the same time, and the second therapeutic agent is selected from the group consisting of anti-virus agents, anti-cancer agents, anti-inflammation agents, and immune improvement agents. The second agent can be taken prior to, simultaneously or after the administration of the pharmaceutical composition of the present invention.

As above mentioned cosmetic products contain the Antrodia camphorata cultivated by the method of the present invention, can be applied to skin cares for the purposes of whitening, anti-aging and anti-wrinkling. According to the preferred embodiment of the present invention, the above mentioned cosmetic products further include carriers. The carrier is selected from the group consisting of color make-ups (such as foundation, blusher and lip sticks), hair products (such as hair dye, shampoo, and conditioner), underarm deodorants, lady perfume, male toilet water, lotion and other skin care products. Furthermore, the above mentioned cosmetic products may further include fragrances or color additives, selected from a group consisting of FD&C red No. 40, FD&C red No.3, FD&C blue No.2, FD&C blue No.No.1, FD&C green No.1, FD&C green No.3, FD&C yellow No.6 or FD&C yellow No.5.

As above mentioned soft drinks containing the Antrodia camphorata cultivated by the method of the present invention, according to the preferred embodiment, further comprise a health food ingredient selected from the group consisting of citric acid, taurine, vitamin C, vitamin B group, pantothenic acid, pantothenate, nicotinic acid, nicotinate, inositol, carotene, lysine and the combinations thereof. In addition, the soft drink further includes a food additive, and the additive is selected from the group consisting of carbonated water, granulated sugar, fructose, natural seasonings, food pigments and the combinations thereof. Furthermore, the soft drinks are allowed to further include flavor enhancers.

As above mentioned fermentation method to produce the alcoholic products from the cultivated Antrodia camphorata, anti-oxidation potency of the alcoholic products from the cultivated Antrodia camphorata is 2.5-4.0 times relative to the non-fermented products from the cultivated Antrodia camphorata.

According to the preferred embodiment, the fermentation method includes a pre-cultivation process for the Antrodia camphorata on a slope surface, following by transferring a portion of pre-cultivated Antrodia camphorata into a liquid culture medium. Thereafter, the Antrodia camphorata cultivated in the liquid culture medium is transferred to a solid culture medium. The ferment is added for fermentation and the cultivation carries on for 4-6 weeks. The alcoholic product of the fermented Antrodia camphorata is filtered. The fermented alcoholic product from Antrodia camphorata is proved to effectively inhibit the growth of human cancer cell. In addition, the auxiliary activities to inhibit the growth of human cancer cells, such as breast cancer cells (such as MCF-7), liver cancer cells (such as Hep 3B), prostate cancer cells (such as LNCaP), and the other cancer cells are also proved. Furthermore, the inhibition for the oxidation of the human low density lipoprotein, the reduction for the atherosclerosis and the recovery of the liver damage are also discovered.

After the Pasteurization, the alcoholic extract obtained from the fermented Antrodia camphorata contains 3.5%-16% of alcohol. If distillation process is carried on, the alcohol concentration of the extract can be increased to about 20%-40%.

The cultivation method for the Antrodia camphorata of the present invention reduces the time frame with mass production to grow the Antrodia camphorata. Furthermore, the antioxidant, anti-proliferation effect and the recovery for damaged liver cell by the cultivated Antrodia camphorata is proved to be similar as the wild Antrodia camphorata. Therefore, the cultivated Antrodia camphorata is applied to medicinal products, consumer products, cosmetics, health care products and fermentation applications, such as, the cultivated Antrodia camphorata is applied to inhibit the prostate cancer and abnormal proliferation, anti-oxidation low density lipoprotein, prevention of atherosclerosis and the recovery of liver function. The cultivated Antrodia camphorata is treated as additive to medicine, foods, skin cares and cosmetic make ups, hair products, drinks and beverages.

It is to be understood that the foregoing general description and the following detailed description are exemplary and explanatory only and are not restrictive of the invention, as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a preferred embodiment illustrating a simplified schematic diagram for the Antrodia camphorata cultivation method of the present invention.

FIG. 2 illustrates the anti-oxidation effects of the extracts from Antrodia camphorata with various cultivated periods based on the anti-LDL oxidation Model.

FIG. 3 illustrates the anti-oxidation effects of the extracts from Antrodia camphorata with various carbon sources for cultivation, based on the anti-LDL oxidation Model.

FIG. 4 illustrates the anti-cancer effects of the extracts from Antrodia camphorata with various cultivated periods, based on the human cancer cell line Model.

FIG. 5 illustrates the anti-cancer effects of the extracts from Antrodia camphorata with various carbon sources for cultivation, based on the human cell line Model.

### DESCRIPTION OF THE EMBODIMENTS

Reference is made in detail to embodiments of the invention. While the invention is described in conjunction with the embodiments, the invention is not intended to be limited by these embodiments. On the contrary, the invention is intended to cover alternatives, modifications and equivalents, which may be included within the spirit and scope of the invention as defined by the appended claims. Furthermore, in the following detailed description of the invention, numerous specific details are set forth in order to provide a thorough understanding of the invention. However, as is obvious to one ordinarily skilled in the art, the invention may be practiced without these specific details. In other instances, well-known methods, procedures, components, and circuits have not been described in detail so that aspects of the invention will not be obscured.

The cultivation method:

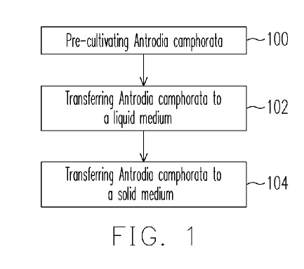

FIG. 1 is a preferred embodiment illustrating a simplified schematic diagram for the Antrodia camphorata cultivation method of the present invention. Referring to FIG. 1, a pre-cultivation process for the Antrodia camphorata (step 100) is performed. The strain is either a wild Antrodia camphorata strain or a collected or cultivated Antrodia camphorata strain by, for example, collecting the fruit body of Antrodia camphorata or a colonized part of Antrodia camphorata from the duramen of Cinnamomum kanehirai. The collected strain from the fruit body of Antrodia camphorata or the colonized part of Antrodia camphorata from the duramen of Cinnamomum kanehirai, is raised in 3% of water agar and stored in incubator at 25° C. Once the mycelium has grown up, a cutting process to separate each hypha is performed to transfer the separated hypha on a slope surface of Potato dextrose agar (PDA) or other suitable cultivation medium. This pre-cultivated purpose is to preserve the strain, any skills in the art is accepted to be applied.

After strain confirmation of the separated spore, hypha, and colonized type, the confirmed strain is transferred into a liquid cultivation medium (step 102). The process is performed by collecting a portion of pre-cultivated Antrodia camphorata by step 100 into liquid culture medium for further cultivation. This step is accepted to apply any culture medium used by skills in the art for Antrodia camphorata, such as, the liquid cultivation disclosed in U.S. Pat. No. 6,355,475. In one preferred embodiment, the pH value for the liquid cultivation process is, for example, pH 4˜7, with the cultivation period ranging from 7˜28 days.

The next process (step 104) is to transfer the liquid state cultivated Antrodia camphorata to a solid cultivation medium for further cultivation. In one preferred embodiment, the ratio between the liquid cultivation medium and the solid cultivation medium are ranged between 1:1 to 3:1.

In particular, the solid cultivation medium includes a substrate, a carbon source, a nitrogen source and at least one of inorganic salts. The above mentioned substrate is selected from the group consisting of rice, agar, or a mixture of rice and agar. In one of the embodiment, the rice is selected from the group consisting of Glutinous Rice, Japonica Rice, Indica Rice, or Zizania (also called Water Rice or Wild Rice).

In another preferred embodiment, the substrate is consisted of rice and agar, and the weight percentage of rice is ranged from 0% to 3%, as the total weight of the solid culture medium being 100%.

In addition, the above mentioned carbon source for the solid culture medium is selected from the group consisting of glucose (also as dextrose), fructose, galactose, lactose, starch, cellulose, sucrose and the combinations; the above mentioned nitrogen source for the solid culture medium is selected from the group consisting of peptone, tripeptone, beef extract, malt extract, yeast extract and the combinations thereof.

In one preferred embodiment, the ratio between the carbon source and the nitrogen source are ranged between 1:1 to 5:1. The preferred weight percentage of the carbon source is ranged from 0.5% to 10%, as the total weight of the solid culture medium being 100%.

The preferred weight percentage of the nitrogen source is ranged from 0.05% to 5%, as the total weight of the solid culture medium being 100%.

Furthermore, the above mentioned inorganic salts are selected from the group consisting of phosphate, sodium chloride (NaCl), magnesium sulfate (MgSO₄), potassium chloride (KCl); and the phosphate is selected from a group consisting of di-potassium hydrogen phosphate (K₂HPO₃), tri-potassium phosphate (K₃PO₃), potassium di-hydrogen phosphate (KH₂PO₃), sodium di-hydrogen phosphate (NaH₂PO₃), di-sodium hydrogen phosphate (Na₂HPO₃); the concentrations of the inorganic salts are ranged from 0.01% to 2.5%, based on the total 100 of weight percentage.

The temperature for the solid cultivation medium of the Antrodia camphorata is ranged from 18 to 28° C. (occasionally even up to 30° C). In addition, sunshine and vibration are avoided during the solid state cultivation. The cultivation period of the solid state cultivation is, for example, 20-90 days.

The above mentioned method is used to cultivate the mycelium of Antrodia camphorata. The mycelium of the cultivated Antrodia camphorata provides the similar biological activities as the fruit body of the wild Antrodia camphorata. Similarly, it can be used for inhibiting human cancer cell growth, lowering the possibility of atherosclerosis by inhibiting the oxidation of human LDL, and recovering the damaged liver functions.

The following paragraphs describe the testing results for the mycelium of Antrodia camphorata cultivated by the present invention, to show that the mycelium of Antrodia camphorata can be effective for inhibiting cancer cell growth and preventing the oxidation of LDL.

Tests of Anti-Oxidation Activity

The anti-oxidation activity test is performed by obtaining the human low density lipoprotein (LDL) by high speed centrifugal separation and inducing oxidation of human LDL by copper ion (Cu²⁺) at 10 μM. Testing samples are added at the same time to perform the LDL oxidation tests. The conjugated dienes in the LDL is used as a marker to monitor the oxidation process by continuous observing the UV spectrum at 234 nm absorption. In addition, the comparing standard for anti-LDL oxidation test is the derivative (Trolox) of vitamin E at 2 μM as positive control.

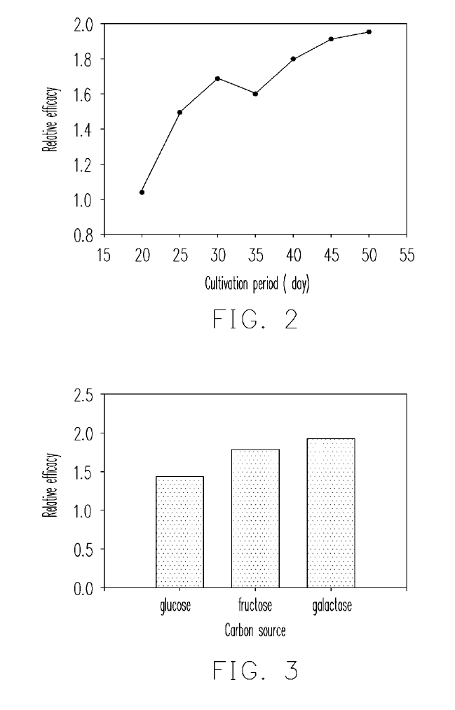

Table 1 lists the anti-LDL oxidation efficacy of the extracts from Antrodia camphorata that is cultivated for various cultivation periods, and oxidation of LDL is induced by copper ions. The first column in Table 1 shows the cultivation periods in days. FIG. 2 shows the relationship between the cultivation periods and anti-LDL oxidation potency, based on Table 1.

TABLE 1
Anti-oxidation tests in the concentration of 1 μL/mL Antrodia camphorata
extracts

| Cultivation periods for Antrodia | |
|---|---|
| camphorata (days) | Relative potency |
| 20 | 1.0 |
| 25 | 1.5 |
| 30 | 1.7 |
| 35 | 1.6 |
| 40 | 1.8 |
| 45 | 1.9 |
| 50 | 1.9 |

PS: Trolox is the derivative of Vitamin E and is treated as comparison standard, based on the Trolox potency defined to be “1.00” for the effective concentration at the concentration of 2.00 μM.

According to Table 1 and FIG. 2, the longer the cultivation period is, the better anti-LDL oxidation potency the Antrodia camphorata cultivated of the present invention provides.

In addition, further tests are performed for the anti-LDL oxidation potency of the extracts of Antrodia camphorata cultivated in different carbon sources . Table 2 lists the carbon sources which are used to cultivate Antrodia camphorata.

Table 2 lists the outcomes of anti-LDL oxidation efficacy by Antrodia camphorata extracts, which are cultivated on the solid culture medium with various carbon sources. FIG. 3 illustrates the relationship between the various carbon sources and corresponding anti-LDL oxidation efficacy of Antrodia camphorata, based on Table 2.

TABLE 2
Anti-oxidation tests in the concentration of 1 μL/mL Antrodia
camphorata extracts

| Carbon source | Relative potency |
|---|---|
| Dextrose | 1.4 |
| Fructose | 1.8 |
| Galactose | 2.0 |

PS: Trolox is the derivative of Vitamin E and is treated as comparison standard, based on the Trolox potency definded to be “1.00” for the effective concentration at the concentration of 2.00 μM.

According to the results shown in Table 1 and Table 2, the cultivated mycelium of Antrodia camphorata indeed shows the anti-oxidation effects for human low density lipoprotein (LDL).

Inhibition Tests of Human Cancer

The test model designed by National Cancer Institution, (NCI) for screening anti-cancer drugs is used herein. Human cell lines, such as, human prostate cancer cells (such as LNCaP), human liver cancer cells (such as Hep 3B) and human breast cancer cells (such as MCF-7), are used to monitor in vitro studies. In the tests, cell lines of Human liver cancer cells (such as Hep 3B) and breast cancer cells (such as MCF-7) are cultivated in the medium comprising calf serum for 24 hours and then transferred to a new medium. The samples are added and then the cell lines are cultivated for 48 hours. The cell survival rates are evaluated by MTT staining analysis. In addition, human prostate cancer cell line (LNCaP) is cultivated in the medium comprising calf serum for 24 hours and then transferred to a new medium. Then, samples are added and the cell lines are cultivated for 72 hours. The cell survival rates are evaluated by MTT staining analysis.

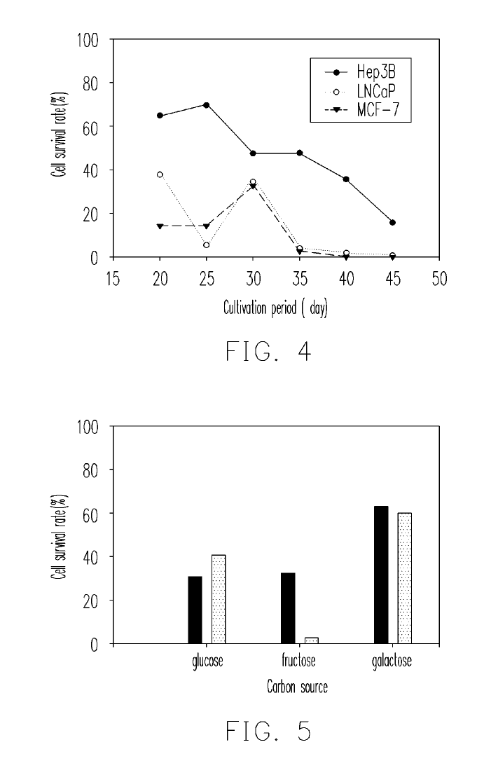

Table 3 lists the cancer cell inhibiting effects of the extracts from the cultivated Antrodia camphorata with various cultivation periods in the solid culture medium. In addition, FIG. 4 illustrates the relationship between the extracts cultivated by various cultivation periods and the survival rates of cancer cells based on Table 3.

TABLE 3
Anti-cancer activity tests at the concentration of 10 μL/mL for Antrodia
camphorata extracts

| Cultivation periods for | | | |
|---|---|---|---|
| Antrodia camphorata | Cell survival rate (%) | | |
| (days) | Hep 3B | LNCaP | MCF-7 |
| 20 | 64 | 37 | 14 |
| 25 | 68 | 4.9 | 13 |
| 30 | 46 | 33 | 31 |
| 35 | 46 | 3.5 | 2.5 |
| 40 | 33 | 2.0 | 0.0 |
| 45 | 13 | 0.74 | 0.0 |

PS:

1. The tests are conducted in 96 wells culture plate to evaluate the survival rate of cancer cells, through MTT staining analysis.

2. MTT staining analysis: MTT, one kind of tertrazolium salt, is 3-[4,5-Dimenthylthialzol-2-yl]2,5-diphenyltetrazolium bromide. It is a yellow dye, which can be absorbed in living cells, and be reduced into blue-colored formazan by succinate-tetrazolium reductase in mitochondria, as the common screening method to evaluate the cell growth and proliferation.

According to the outcome from the Table 3 and FIG. 4, the longer the cultivation periods, the better inhibition effects are provided toward the growth of human prostate cancer cells.

In addition, tests are performed on extracts from Antrodia camphorata cultivated by various carbon sources to inhibit the growth of human cancer cells. Table 4 lists the efficacy of the extracts from Antrodia camphorata cultivated by various carbon sources in the solid medium (the cultivation period is 40 days), to show the survival rates of human cancer cells. FIG. 5 illustrates the relationship between the extracts from Antrodia camphorata by various carbon sources and the survival rates of cancer cells, based on figures and Table 4.

TABLE 4
Anti-cancer activity tests at the concentration of 10 μL/mL of Antrodia
camphorata extracts

| | Cell survival rate (%) | | |
|---|---|---|---|
| Carbon sources | Hep 3B | LNCaP | MCF-7 |
| Dextrose | 32 | 42 | 30 |
| Fructose | 33 | 2.0 | 0.0 |
| Galactose | 64 | 60 | 21 |

According to Table 4 and FIG. 5, all three extracts from various carbon sources have growth inhibition effects on cancer cells. In particular, the extract from Antrodia camphorata cultivated by fructose has the best anti-cancer effect.

Liver Cell Protection

This test model based on the publication of the journal Cancer Research is used to screen the liver cell protection medicines (J-K Lin and C-K Chou, In Vitro Apoptosis in the Human Hepatoma Cell Line Induced by Transforming Growth Factor β1, Cancer Res., 1992, 52, 385-388). The test method applies transformation growth factor β (TGF-β) for stimulating apoptosis as the screening model in vitro studies. Human hepatoma cell line (Hep 3B) is cultivated in the medium comprising calf serum for 24 hours, and then transferring to a new medium. After adding 2 ng/mL of transformation growth factor β (TGF-β) and samples to be tested, the cell line is cultivated for another 48 hours. Finally, MTT staining analysis is applied to evaluate the survival rate, and then the survival rate is converted into the recovery rate.

TABLE 5
Liver cell protection tests at the concentration of 10 μL/mL of Antrodia
camphorata extracts

| Cultivation periods for Antrodia | |
|---|---|
| camphorata (days) | Liver cell recovery rate (%) |
| 20 | 147 |
| 25 | 129 |
| 30 | 158 |
| 35 | 122 |
| 40 | 66.0 |

Table 5 lists the protection effects of the Antrodia Camphorata extracts cultivated by various cultivation periods toward the apoptosis of Hep 3B cell line stimulated by transformation growth factor β (TGF-62 ). The outcome shows the Antrodia camphorata extracts by the cultivated periods of around 20˜40 days provide the recovery effects for the TGF-β stimulated apotosis or damage of Hep 3B cell line.

The Cultivation and Fermentation of Antrodia Camphorata for the alcoholic products (wine & liquod)

According to a preferred embodiment, cultivation and fermentation of Antrodia camphorata for the alcoholic extracts follows the similar method described above. Referring to FIG. 1, a pre-cultivation process for the Antrodia camphorata is performed on a sloped surface (step 100), and a portion of the pre-cultivated Antrodia camphorata is collected and transferred to a liquid medium for further cultivation (step 102). The Antrodia camphorata cultivated in the liquid medium is transferred to a solid medium (step 104) for further cultivation. It is noted that fermentation is performed at the same time along with the cultivation by adding a ferment in (step 104) and cultivating for another 4-6 weeks. Thereafter, the fermented Antrodia camphorata containing alcohol is filtered and pasteurized. The filtered product (the alcoholic product) of the fermented Antrodia camphorata contains about 3.5%-16% of alcohol. If a distillation process is performed, the distilled product of the fermented Antrodia camphorata may contain 20%-40% of alcohol and can be commercialized as a liquor.

According to Table 6, the anti-oxidation effects for the non-fermented extract of Antrodia camphorata, the filtered product of the fermented Antrodia camphorata (1), the distilled product of the fermented Antrodia camphorata (2) are tested, and the data indicated that anti-oxidation potency products of the extracts from the fermented Antrodia camphorata is 2.5˜4.0 times relative to the non-fermented extract. The sweetness of the distilled alcoholic product from the fermented Antrodia camphorata (2) is 6 and the pH value is 3.75.

TABLE 6
The anti-oxidation tests for the extracts from the fermented Antrodia
Camphorata

| | | Activity compared to |
|---|---|---|
| | | 2.0 μM of Trolox |
| | Relative efficacy | (μL/mL) |
| non-fermented extract from | 2.3 | 0.98 |
| Antrodia camphorata | | |
| filtered product of fermented | >5.6 | 0.24 |
| Antrodia camphorata (1) | | |
| distilled product of fermented | >5.6 | 0.38 |
| Antrodia camphorata (2) | | |

PS:

1. Trolox is the derivative of Vitamin E and is used as a standard for comparison, the potency of Trolox defined to be “1.0” at a Trolox concentration of 2.00 μM.

2. The concentrations of samples are 2.0 μL/mL.

The alcoholic products from the Antrodia camphorata cultivated by the method of the present invention may be used as pharmaceutical compositions for providing inhibitory effects in the growth of human cancer cells, including breast cancer cells (such as MCF-7), liver cancer cells (such as Hep 3B), prostate cancer cells (such as LNCaP), and the other cancer cells. The alcoholic product from the Antrodia camphorata can provide anti-oxidation effects in human low density lipoproteins for reducing the occurrence of atherosclerosis.

Applications of the Cultivated Antrodia Camphorata

According to the above descriptions, the present invention successfully cultivates the mycelium of Antrodia camphorata. Compared to the traditional artificial cultivation method, the method of the present invention performs the solid state cultivation after the liquid medium cultivation, suitable for mass production of the mycelium of Antrodia camphorata. In addition, the test outcomes prove the cultivated mycelium of Antrodia camphorata in this invention having the similar biological effects as the wild Antrodia camphorata, i.e. having similar biological potency in the inhibition of cancer cell growth and anti-oxidation effects. Therefore, the mycelium of Antrodia camphorata cultivated by the present invention can be applied to for pharmaceutical, food, cosmetic and prophylaxis purposes. Details for the applications are described as follows.

In one embodiment, the mycelium of Antrodia camphorata is applied in the food industries. Accordingly, the present invention is directed to a food composition including the Antrodia camphorata cultivated by the method of the present invention. The form of the food composition is accepted as a powder, gel, solid or liquid. In one preferred embodiment, the food composition further includes an additive and the additive is selected from the group consisting of a Chinese herb, a health food ingredient, or a food ingredient. The above mentioned health food is selected from the group consisting of citric acid, taurine, vitamin C, vitamin B group, pantothenic acid, pantothenate, nicotinic acid, nicotinate, inositol, carotene, lysine and the combinations. The above mentioned food ingredient is selected from the group consisting of vegetables, fruits, meat and the combinations thereof. For each ingredient, the mixing ratio is designed to fulfill related regulations and allowed for adjustments. Even directly mixing the Antrodia camphorata of the present invention into a food composition is acceptable. Therefore, the mixing ratios for the food composition are not limited by the present invention.

As the mycelium of the cultivated Antrodia camphorata has the effects of anti-oxidation and anti-cancer growth, the food composition comprising the cultivated Antrodia camphorata of the present invention is classified as a health food.

In another embodiment, the mycelium of Antrodia camphorata cultivated by the present invention is further applied for pharmaceutical products. Therefore, the present invention is directed to a pharmaceutical composition comprising the Antrodia camphorata cultivated by the method of the present invention. The form of the pharmaceutical composition is selected from the group consisting of tablet, powder, granular, capsule, rapid (oral integrated) tablet, injection, lyophilized injection, suspension, emulsion, syrup, tincture or solution. For each ingredient, the mixing ratio is designed to fulfill related regulations and allowed for adjustments. Even directly mixing the cultivated Antrodia camphorata of the present invention in a pharmaceutical composition is acceptable. Therefore, the mixing ratios for the pharmaceutical compositions are not limited by the present invention.

As the mycelium of the cultivated Antrodia camphorata has the effect in anti-cancer growth, the pharmaceutical composition comprising the cultivated Antrodia camphorata of the present invention can be applied for cancer therapy. As the mycelium of the cultivated Antrodia camphorata has the anti-oxidation effect, the pharmaceutical composition of the present invention can be applied to treat atherosclerosis.

In another embodiment, the mycelium of Antrodia camphorata cultivated by the present invention is further applied in cosmetic products for the purposes of whitening, anti-aging, and anti-wrinkling. Therefore, the present invention is directed to a cosmetic product comprising the Antrodia camphorata cultivated by the method of the present invention. In one preferred embodiment, the cosmetic product further includes a carrier, and the carrier is selected from the group consisting of color make-ups, hair products, underarm deodorants, lady perfumes, male toilet water, lotions and other skin care products. The above mentioned color products are, for example, a foundation, blusher and lipsticks. The above mentioned hair products are, for example, a hair dye, shampoo, conditioner . . . etc. In one preferred embodiment, the above mentioned cosmetic products further include a color additive. The color additive is selected from a group consisting of FD&C red No. 40, FD&C red No.3, FD&C blue No.2, FD&C blue No.1, FD&C green No.1, FD&C green No.3, FD&C yellow No.6 or FD&C yellow No.5. In one preferred embodiment, the cosmetic product further includes a fragrance. For each ingredient, the mixing ratio is designed to fulfill related regulations and allowed for adjustments. Even directly mixing the Antrodia camphorata of the present invention into the cosmetic product is acceptable. Therefore, the mixing ratios for the cosmetic products are not limited by the present invention.

As the mycelium of the cultivated Antrodia camphorata has the anti-oxidation effect, the cosmetic product of the present invention can be applied for anti-aging purposes.

In anther embodiment, the mycelium of Antrodia camphorata cultivated by the present invention is further applied in soft drinks. Therefore, the present invention is directed to a soft drink comprising the Antrodia camphorata cultivated by the method of the present invention. In one preferred embodiment, the soft drink further includes a carrier, and the carrier is selected from the group consisting of water, bubbly water, alcohol, dairy, juice and the combinations. In one embodiment, the soft drink further includes at least one of the health food ingredients. The above mentioned health food ingredient is selected from the group consisting of citric acid, taurine, vitamin C, vitamin B group, pantothenic acid, pantothenate, nicotinic acid, nicotinate, inositol, carotene, lysine and the combinations. In one preferred embodiment, the composition of the soft drink further includes at least one of the food additives. The above mentioned food additives selected from the group consisting of carbonated water, granulated sugar, fructose, natural fragrance, and food pigment. In one preferred embodiment, the composition of the soft drink further includes a seasoning. For each ingredient, the mixing ratio is designed to fulfill related regulations and allowed for adjustments. Even directly mixing the Antrodia camphorata of the present invention is acceptable. Therefore, the mixing ratios for the soft drink composition are not limited by the present invention.

Due to the effects of anti-oxidation and anti-cancer growth, the mycelium of Antrodia camphorata cultivated by the present invention is added into soft drinks as health drinks.

The above descriptions of specific embodiments of the invention have been presented for purposes of illustration and description. They are not intended to be exhaustive or to limit the invention to the precise forms disclosed. Obviously, many modifications and variations are possible in light of the above teaching. The embodiments were chosen and described in order to explain the principles and the application of the invention, thereby enabling others skilled in the art to utilize the invention in its various embodiments and modifications according to the particular purpose contemplated. The scope of the invention is intended to be defined by the claims appended hereto and their equivalents.

What is claimed is:

1. A method for cultivating Antrodia camphorata, comprising:
pre-cultivating Antrodia camphorata on a sloped surface of a medium,
collecting and transferring a portion of pre-cultivated Antrodia camphorata to a liquid medium, and
transferring Antrodia camphorata cultivated in the liquid medium onto a solid medium and cultivating Antrodia camphorata in the solid medium.

2. According to the method for cultivating Antrodia camphorata as recited in claim 1, wherein the solid medium comprises a substrate, a carbon source, a nitrogen source, and at least one inorganic salt.

3. According to the method for cultivating Antrodia camphorata as recited in claim 2, wherein the substrate is rice.

4. According to the method for cultivating Antrodia camphorata as recited in claim 3, wherein the rice is selected from the group consisting of Glutinous Rice, Japonica Rice, Indica Rice, Zizania (also called Water Rice or Wild Rice) and the combination thereof.

5. According to the method for cultivating Antrodia camphorata as recited in claim 2, wherein the substrate is agar.

6. According to the method for cultivating Antrodia camphorata as recited in claim 2, wherein the substrate is a mixture of rice and agar.

7. According to the method for cultivating Antrodia camphorata as recited in claim 6, wherein a percentage of rice by weight in the mixture is ranged from 0% to 60%, a percentage of agar by weight in the mixture is ranged from 0% to 3%, and a total weight of the solid culture medium being 100%.

8. According to the method for cultivating Antrodia camphorata as recited in claim 2, wherein the carbon source is selected from the group consisting of glucose, fructose, galactose, lactose, starch, cellulose and sucrose.

9. According to the method for cultivating Antrodia camphorata as recited in claim 2, wherein the nitrogen source is selected from the group consisting of peptone, tripeptone, beef extract, malt extract and yeast extract.

10. According to the method for cultivating Antrodia camphorata as recited in claim 2, wherein a ratio of the carbon source and the nitrogen source is ranged from 1:1 to 5:1.

11. According to the method for cultivating Antrodia camphorata as recited in claim 2, wherein a weight percentage of the carbon source is ranged from 0.5% to 10%, and a total weight of the solid culture medium being 100%.

12. According to the method for cultivating Antrodia camphorata as recited in claim 2, wherein a weight percentage of the nitrogen source is ranged from 0.05% to 5%, and a total weight of the solid culture medium being 100%.

13. According to the method for cultivating Antrodia camphorata as recited in claim 2, wherein the inorganic salt is selected from the group consisting of a phosphate, sodium chloride (NaCl), magnesium sulfate (MgSO₄) and potassium chloride (KCl).

14. According to the method for cultivating Antrodia camphorata as recited in claim 2, wherein a concentration of the inorganic salt is ranged from 0.01% to 2.5%, and a total weight of the solid culture medium being 100%.

15. According to the method for cultivating Antrodia camphorata as recited in claim 13, wherein the phosphate is selected from the group consisting of di-potassium hydrogen phosphate (K₂HPO₃), tri-potassium phosphate (K₃PO₃), potassium di-hydrogen phosphate (KH₂PO₃), sodium di-hydrogen phosphate (NaH₂PO₃), di-sodium hydrogen phosphate (Na₂HPO₃).

16. According to the method for cultivating Antrodia camphorata as recited in claim 13, wherein a concentration of the phosphate is ranged from 0.01% to 1.0%, and a total weight of the solid culture medium being 100%.

17. According to the method for cultivating Antrodia camphorata as recited in claim 1, wherein a cultivation temperature of the solid medium is ranged from 18° C. to 28° C.

18. According to the method for cultivating Antrodia camphorata as recited in claim 1, wherein the step of cultivating Antrodia camphorata in the solid medium is free from sun light.

19. According to the method for cultivating Antrodia camphorata as recited in claim 1, wherein the step of cultivating Antrodia camphorata in the solid medium is free from vibration.

20. According to the method for cultivating Antrodia camphorata as recited in claim 1, wherein a cultivation period for the cultivating step in the solid medium is ranged from 20-90 days.

21. According to the method for cultivating Antrodia camphorata as recited in claim 1, wherein a cultivation period for the cultivating step in the liquid medium is ranged from 7-28 days.

22. According to the method for cultivating Antrodia camphorata as recited in claim 1, wherein a pH value for the liquid medium is ranged from 4-7.

23. According to the method for cultivating Antrodia camphorata as recited in claim 1, wherein a weight ratio of the liquid medium and the solid medium is ranged from 1:10 to 1:50.

24. According to the method for cultivating Antrodia camphorata as recited in claim 1, wherein after the cultivating step in the solid medium, mycelia of Antrodia camphorata are obtained.

25. According to the method for cultivating Antrodia camphorata as recited in claim 24, wherein the cultivated Antrodia camphorate is applied to inhibit the growth of a cancer cell.

26. According to the method for cultivating Antrodia camphorata as recited in claim 24, wherein the cultivated Antrodia camphorata contains auxiliary activities to inhibit the growth of cancer cells.

27. According to the method for cultivating Antrodia camphorata as recited in claim 24, wherein the cancer cell is selected from the group consisting of human breast cancer cells, liver cancer cells, prostate cancer cells and other cancer cells.

28. According to the method for cultivating Antrodia camphorata as recited in claim 24, wherein the cultivated Antrodia camphorata contains anti-oxidation effects and is applied to reduce the incidence of atherosclerosis.

29. According to the method for cultivating Antrodia camphorata as recited in claim 24, wherein the cultivated Antrodia camphorata is applied for recovering damaged liver functions.

30. A food composition at least comprising Antrodia camphorata cultivated by the method as recited in claim 1.

31. The food composition as recited in claim 30, wherein a form of the food composition is as powders, a gel, a solid or a liquid.

32. The food composition as recited in claim 30, wherein the food composition further includes an additive, and the additive is selected from the group consisting of a Chinese herb, a health food ingredient, a food ingredient, and the combinations thereof.

33. A pharmaceutical composition at least comprising Antrodia camphorata cultivated by the method as recited in claim 1.

34. The pharmaceutical composition as recited in claim 33, wherein a form of the pharmaceutical composition is selected from the group consisting of tablet, powder, granular, capsule, rapid (oral integrated) tablet, injection, lyophilized injection, suspension, emulsion, syrup, tincture or solution.

35. The pharmaceutical composition as recited in claim 33, wherein the pharmaceutical composition is administered with a second therapeutic agent.

36. The pharmaceutical composition as recited in claim 35, wherein the second therapeutic agent is selected from the group consisting of anti-virus agents, anti-cancer agents, anti-inflammation agents, and immunity improvement agents.

37. The pharmaceutical composition as recited in claim 36, wherein the second therapeutic agent is taken prior to, simultaneously or after administering of the pharmaceutical composition.

38. A cosmetic product at least comprising Antrodia camphorate cultivated by the method as recited in claim 1.

39. The cosmetic product as recited in claim 38, wherein the cosmetic product is applied for whitening, anti-aging, or anti-wrinkling.

40. The cosmetic product as recited in claim 38, wherein the cosmetic product further includes a carrier, and the carrier is selected from the group consisting of color make-ups, hair products, underarm deodorants, lady perfume, male toilet water, lotion and other skin care products.

41. The cosmetic product as recited in claim 40, wherein the make-up is selected from the group consisting of a foundation, a blusher and lip sticks.

42. The cosmetic product as recited in claim 40, wherein the hair product is selected from the group consisting of a hair dye, a shampoo, and a conditioner.

43. The cosmetic product as recited in claim 38, wherein the make-up further includes a color additive.

44. The cosmetic product as recited in claim 38, wherein the color additive is selective from the group consisting of FD&C red No. 40, FD&C red No.3, FD&C blue No.2, FD&C blue No.1, FD&C green No.1, FD&C green No.3, FD&C yellow No.6 or FD&C yellow No.5.

45. The cosmetic product as recited in claim 38, wherein the cosmetic product further includes a fragrance.

46. A soft drink composition at least comprising Antrodia camphorata cultivated by the method as recited in claim 1.

47. The soft drink product as recited in claim 46, wherein the soft drink product further includes a carrier, and the carrier is selected from the group consisting of water, carbonated water, alcohol, a dairy product, a juice and the combination thereof.

48. The soft drink product as recited in claim 46, wherein the soft drink product further includes a healthy food ingredient.

49. The soft drink product as recited in claim 48, wherein the healthy food ingredient is selected from the group consisting of citric acid, taurine, vitamin C, vitamin B group, pantothenic acid, pantothenate, nicotinic acid, nicotinate, inositol, carotene, lysine, and the combinations thereof.

50. The soft drink product as recited in claim 46, wherein the soft drink product further includes a food additive.

51. The soft drink product as recited in claim 50, wherein the food additive is selected from the group consisting of carbonated water, granulated sugar, fructose, natural fragrances, and food pigments.

52. The soft drink product as recited in claim 46, wherein the soft drink product further includes a seasoning.

53. According to the method for cultivating Antrodia camphorata as recited in claim 25, wherein the cancer cell is selected from the group consisting of human breast cancer cells, liver cancer cells, prostate cancer cells and other cancer cells.

54. A fermentation method for an alcoholic product of Antrodia camphorata, comprising:
pre-cultivating Antrodia camphorata on a slope surface of a medium,
collecting and transferring a portion of pre-cultivated Antrodia camphorata to a liquid medium, and
transferring Antrodia camphorata cultivated in the liquid medium onto a solid medium, and adding a ferment to perform fermentation for 4-6 weeks;
filtering and collecting the alcoholic extract of Antrodia camphorata.

55. The method as recited in claim 54, wherein the alcoholic product is applied to inhibit cancer cell growth.

56. The method as recited in claim 54, wherein the alcoholic product contains auxiliary activities to inhibit cancer cell growth.

57. The method as recited in claim 54, wherein the cancer cells are selected from the group consisting of human breast cancer cells, liver cancer cells, prostate cancer cells, and other cancer cells.

58. The method as recited in claim 54, wherein the alcoholic product contains the anti-oxidation effect and is applied to reduce the occurrence of atherosclerosis.

59. The method as recited in claim 54, wherein the alcoholic product is applied for recovering damaged liver functions.

60. The method as recited in claim 54, wherein the filtered alcoholic extract is pasteurized and an alcohol percentage is ranged from 3.5%˜16%.

61. The method as recited in claim 54, wherein the filtered alcoholic product is further distilled and the distilled extract contains an alcoholic percentage ranging from 20%˜40%.

62. The method as recited in claim 54, wherein an anti-oxidation effect of the alcoholic product is 2.5-4.0 times relative to the non-fermented extract.
